SEQUENCE LISTING (3 sequences)

SEQ ID NO: 1 - DNA, 18 bp, Artificial Sequence
  Oligonucleotide primer
gggcaacgtgctggtctg

SEQ ID NO: 2 - DNA, 17 bp, Artificial Sequence
  Oligonucleotide primer
aggcagcctgcactggt

SEQ ID NO: 3 - DNA, 24 bp, Artificial Sequence
  Oligonucleotide primer
ctggcccatcactttggcaaagaa

(19) United States
(12) Patent Application Publication
(10) Pub. No.: US 2010/0092962 A1
(43) Pub. Date: Apr. 15, 2010

(54) Method of Detecting Colorectal Cancer

(76) Inventors: Alexandre Loktionov, Cambridge (GB); Hugo Geoffrey Gifford Lywood, Worcs (GB); Colin George Ferrett, Oxford (GB); Tatiana Bandaletova, Cambridge (GB); Andrew Humphrey Llewelyn, Lyme Regis (GB); Rupert Charles Gifford Lywood, Kidderminster (GB)

Correspondence Address: FOX ROTHSCHILD, LLP;Pittsburgh, 997 Lenox Drive, Bldg. #3, Lawrenceville, NJ, 08648, US

(21) Appl. No.: 12/484,204

(22) Filed: Jun. 13, 2009

Related U.S. Application Data
(63) Continuation-in-part of application No. 11/571,693, filed on Nov. 21, 2007.

(30) Foreign Application Priority Data
Jul. 7, 2004 (GB) 0415277.3

(51) Int. Cl. C12Q 1/68; A61B 10/02

(52) U.S. Cl. 435/6; 600/572

(57) ABSTRACT

A method for predicting the presence of colorectal cancer based on the concentration of DNA in a sample of exfoliated cells collected directly from the surface of the rectal mucosa of a subject. The method consists of quantifying the concentration of DNA in a lysate containing exfoliated cells and correlating the concentration with a range within a statistical model which indicated the likelihood of the presence of colorectal cancer as well as the likely location of the cancer.

### RELATED APPLICATIONS

This application is a continuation-in-part of co-pending U.S. application Ser. No. 11/571,693, filed Jul. 6, 2005, entitled “Colorectal Cell Sampling Device”, which is incorporated by reference in its entirety.

### FIELD OF THE INVENTION

The present invention relates to a method of collecting and analyzing samples of exfoliated cells from the colorectal mucosal surface of a subject, which have been collected with the described device, and to a method of utilizing collected cells to the diagnose the presence of colorectal cancer.

### BACKGROUND OF THE INVENTION

Sporadic colorectal cancer (CRC) is one of the most frequently occurring and deadly of the oncological diseases affecting people in developed Western countries and is the second most prevalent malignancy worldwide, being the second commonest cause of cancer-related death. It predominantly affects people over the age of 50.

A serious obstacle to early diagnosis of CRC is the absence of early, readily identifiable clinical manifestations in the majority of cases. It is only in the advanced stages of the disease, when larger tumours have formed which result in pain, bleeding and symptoms of obstruction, that the disease is readily diagnosed. However, the late stages of the disease are also associated with invasive or metastatic tumours and high incidence of death. Thus, detection of colorectal tumours prior to the advanced stages of the disease would greatly increase the chances of successful surgical intervention and overall survival rates.

The benefits of CRC screening are generally recognized. Two categories of screening approaches are defined in the latest US CRC screening guidelines. The first category includes faecal tests such as faecal occult blood test (FOBT), faecal immunochemical test (FIT) and stool DNA analysis. The second category includes full or partial structural exams such as colonoscopy, flexible sigmoidoscopy (FS) and imaging techniques including computed tomographic colonography (CTC) and double contrast barium enema (DCBE).

The first category of tests is inexpensive and simple, however, they are known to produce unacceptably high rates of both false negative and false positive results. Additionally, all positive findings on faecal tests, DCBE, CTC and FS require a confirmatory colonoscopy, which remains the key element of CRC diagnosis. Despite the limitations, faecal tests are presently the screening method of choice.

The second category of tests, namely full or partial structural examinations, are regarded as precise and reliable diagnostic procedures, however, their invasiveness, cost and the requirement for skilled and experienced specialists to carry out the procedures make their use in routine screening impractical. These tests also entail the cumulative risk of complications, especially if applied in elderly patients or repeatedly performed for screening purposes. Concerns about cost, required specialist skill and patient radiation exposure are also true for the recently-introduced computed tomographic colonography (virtual colonoscopy).

None of the methods in common use is able to provide a combination of low invasiveness, simplicity and affordability with high sensitivity and specificity. As a result, unnecessary colonoscopies remain common, and the search for more efficient CRC screening methods continues.

Alternative approaches to diagnosing CRC based upon a direct indicator of tumour presence have also been investigated. One indicator that has been identified is the analysis of exfoliated colonocytes. Exfoliation of colonocytes (i.e. the spontaneous detachment of cells from the epithelial layer of the colonic mucosa) is an important cell renewal mechanism in the human gut. Cytological analysis of colonocytes obtained from colonic or rectal washings (i.e. by irrigation of the colorectal mucosa) is well known in the art and has shown that morphologically distinct exfoliated neoplastic cells could be detected in CRC patients. However, the method of obtaining these samples, namely an invasive colonic lavage procedure, suffers from the same disadvantages as sigmoidoscopy/colonoscopy, and requires detailed cytological analysis of the sample once it is obtained.

The prevailing approach to obtaining samples of exfoliated epithelial cells has been to isolate them from human faeces. Human faeces were identified as a source of such cells, as the exfoliated cells of the colonic epithelium can be excreted in conjunction with other faecal matter. However, purification of high quality human DNA from faeces is challenging and normal squamous epithelium of the anal canal shed during the act of defecation contributes to the yield of DNA isolated from stool, thus decreasing cancer detection efficiency.

A number of attempts have been made to develop methods to isolate colonocyte-derived genetic material (DNA) from human faeces to develop diagnostic procedures employing molecular biomarkers of malignancy. While DNA directly isolated from homogenized faeces can be amplified and analysed for the presence of cancer-associated genetic alterations, the absence of a highly reliable single molecular biomarker for cancer results in the use of multiple molecular markers reflecting a number of genetic alterations known to be present in malignant cells at relatively high frequencies.

Although detection of colorectal tumours by multi-target molecular assays appears to be possible, the feasibility of these methods for screening purposes remains questionable due to the high cost and relative complexity of laboratory procedures involved.

Although work utilizing exfoliated colonocytes was initially geared to the development of a molecular diagnostic assay for CRC, it has been found that a quantitative analysis of colonocyte-derived DNA from human stool surface could be used for CRC diagnosis and screening because the relative DNA concentration in subjects having CRC was much higher compared to healthy individuals. It has been shown that there exists a striking difference between CRC patients and healthy individuals employing a calculated index relating to the amount of DNA extracted from cells isolated from the stool surface to stool weight (stool DNA index or SDNAI).

It is well known in the art that mucocellular layer covering the human rectal mucosa is particularly rich in well-preserved exfoliated colonocytes. In addition, the cellular content of this layer in CRC patients appears to be much higher than in healthy individuals primarily due to greatly increased presence of malignant colonocytes. Therefore, the tumour cells of CRC patients, which are apoptosis-resistant and much better adapted to autonomous existence should quantitatively dominate the rectal exfoliated cell pool. Clinical observations describing distal (e.g. anal) implantation of persisting exfoliated cells from removed colorectal tumours strongly corroborate this hypothesis.

There is thus a great need for a method of direct collection of exfoliated epithelial cells from the surface of rectal mucosa without the problems of material loss and serious contamination with other tissue elements, and a method of extracting and quantifying the DNA present in these cells for purposes of diagnosing CRC.

### SUMMARY OF THE INVENTION

According to a first aspect of the invention, there is provided a colorectal cell sampling device designed to collect exfoliated colonocytes directly from the surface of the rectal mucosa, thereby eliminating or greatly reducing the likelihood of faecal contamination of the sample.

The device overcomes the difficulties with digital sampling by holding a flexible membrane within an insertion member both on insertion and withdrawal such that material loss is avoided and such that the sample is not contaminated by cells from other surfaces (e.g. the squamous epithelium of the anal canal) or from faecal matter present in the colon.

In addition, by sampling the mucosal surface directly, the device overcomes the difficulties associated with whole stool sampling including the unpleasant nature of the work, the low concentration of cells obtainable, the high levels of contamination with faecal matter (especially bacteria), and difficulties in standardization related to such problems, for example, great variability of stool size and consistency.

Although the device is invasive, it is far less invasive than the devices currently used for colonoscopy/sigmoidoscopy, and does not require operation by a skilled and highly trained operator. The device may even be self-administered by the subject. The reduced level of invasiveness and the absence of complication risk are likely to lead to greater patient acceptance. These advantages should in turn allow for more sampling to be carried out, and at a lower cost.

The device consists of an anal insertion member to which is attached a flexible membrane having an inner, cell-sampling surface. Pressurization of the cavity of the insertion member will cause the flexible membrane to inflate, and turn inside-out, thereby allowing the cell sampling surface to contacting the rectal mucosa. After collection, the flexible membrane is withdrawn into the body of the insertion member, and the insertion member sealed for transport to a laboratory.

The flexible membrane, when withdrawn into the interior of the rigid body of the insertion member, forms a receptacle such that fluid may be added, such as reagents or lysis buffers. The insertion member may be adapted to engage with a sealing means to seal the flexible membrane and any added liquid within the body of the insertion member, thereby allowing the sampling device, containing the sample, to be stored and transported prior to further analyses being carried out on the sample.

In a second aspect of the invention, a technique based on the measurement of the concentration of DNA isolated from exfoliated material collected from the surface of the rectal mucosa has been developed as a simple screening test for colorectal disease. High DNA scores clearly correlate with the presence of colorectal cancer and inflammatory bowel disease, indicating that the test can also be used in symptomatic subjects for identifying cases requiring urgent attention as well as in non-symptomatic subjects.

The method of screening for and diagnosing colorectal cancer comprises utilizing the described device to collect a sample of exfoliated cells from the rectal mucosa, recovering the collected sample from the sampling device and performing an analysis on the sample.

In a preferred embodiment of the invention, the analysis is selected from DNA quantification, DNA extraction followed by its quantification and optional molecular analysis, cytological/cytochemical investigation and biochemical tests. It is to be noted that the accuracy of screening by any of these methods will be improved by the provision of a sample with low levels of contaminants.

### BRIEF DESCRIPTION OF THE FIGURES

The invention will now be described, by way of example only, with reference to the accompanying drawings in which:

FIG. 1 shows a cross-sectional view of a cell sampling device of the invention.

FIG. 2 shows a schematic representation of a cell sampling device of the invention wherein the means for pressurisation comprise a syringe.

FIG. 3 shows a schematic representation of a cell sampling device of the invention wherein the means for pressurisation comprise a source of compressed air.

FIG. 4 shows the components required for sampling exfoliated cells from a colorectal mucosal surface of a human subject.

FIG. 5 shows an example of a method of sampling exfoliated cells from a colorectal mucosal surface of a human subject using any of the devices shown in FIGS. 1-4.

FIG. 6 shows an example of the steps which may follow the method depicted in FIG. 5.

FIG. 7 shows an embodiment of the cap design having threaded inner and outer surfaces.

FIG. 8 shows the use of the double threaded cap design of FIG. 7.

FIGS. 9a and 9b show the result distribution patterns for the PicoGreen assay method and the QRTPCR assay method respectively.

FIGS. 10a-10f show ROC curves describing sensitivity and specificity of detection of CRC by DNA score assessment:
(A) both proximal and distal tumours (53 eligible cases) by PicoGreen;
(B) (both proximal and distal tumours (53 eligible cases) by QRTPCR;
(C) proximal tumours only (18 eligible cases) by Pico Green;
(D) proximal tumours only (18 eligible cases) by QRTPCR;
(E) distal tumours only (35 eligible cases) by Pico Green;
(F) distal tumours only (35 eligible cases) by QRTPCR.

FIGS. 11 and 12 show specificity and sensitivity percentages for cut off points of 2.5 μg/ml and 5.0 μg/ml respectively.

### DETAILED DESCRIPTION OF THE INVENTION

Description and Use of the Cell Sampling Apparatus

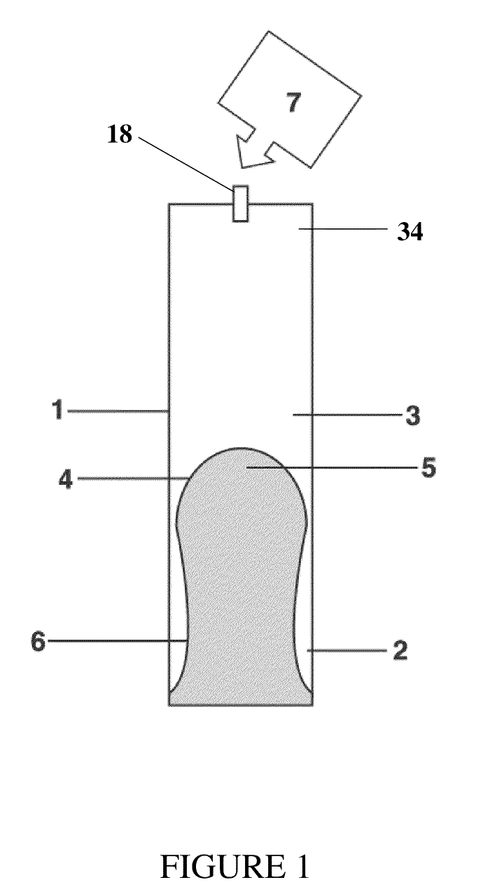

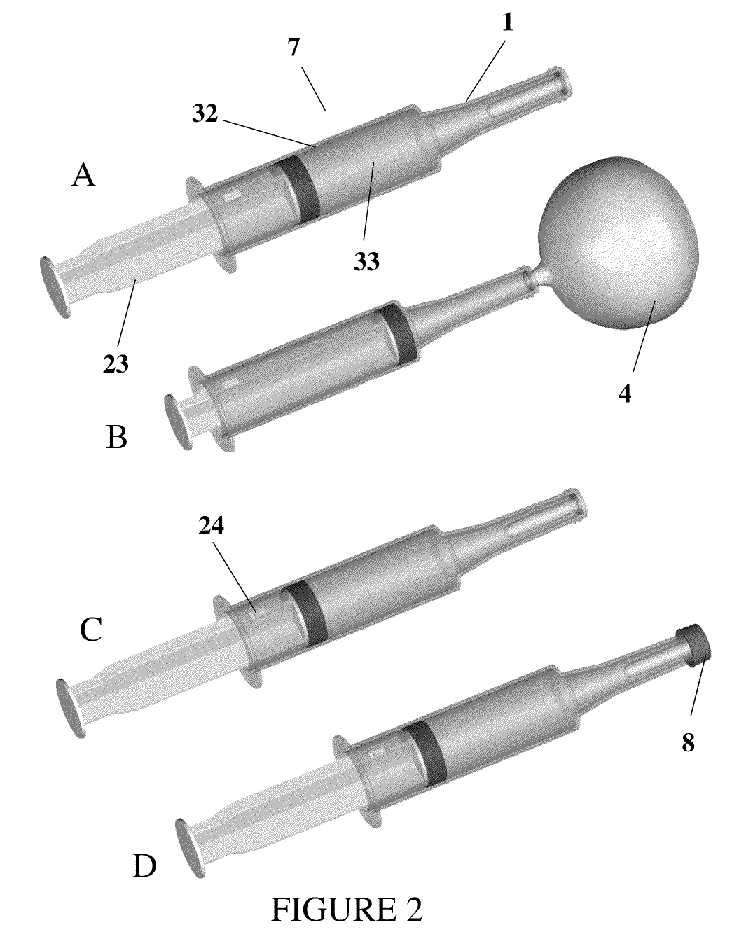

The cell sampling device of FIG. 1 is designed for insertion into a rectal cavity. The device comprises a substantially cylindrical insertion member 1 with an interior cavity 3, closed at the distal insertion end 2 by a flexible and resilient membrane 4 which is sealingly attached to the member 1 at the distal end 2. In the position shown in FIG. 1, the membrane 4 is held within the cavity 3, and is adapted to emit from the cavity 3 when the cavity 3 is pressurised by means 7 (shown in more detail in FIG. 2). The membrane 4 has a cell sampling surface 5 which in the rest position shown in FIG. 1 is the inner surface, but when the membrane emits is the outer surface, and an opposing surface 6 which in the rest position is the outer surface, but which becomes the inner surface when the membrane emits. The membrane may be made of nitrile, latex or a rubber based substance. At the proximal end 34, the cavity 3 is closed by a self-sealing valve 18, to which the pressurisation means 7 is adapted to be attached.

The embodiment of the invention wherein the means for pressurisation of the interior cavity 7 is an integrated syringe is shown in FIG. 2 which schematically also shows the steps necessary to sample exfoliated cells from a colorectal mucosal surface of a human subject (FIGS. 2A-2D).

FIG. 2A shows a representation of the cell sampling device prior to insertion into a rectal cavity. The syringe 7 is attached to an insertion member 1 substantially as described in FIG. 1. The syringe has a plunger 23 which sealingly slides along a barrel 32 of the syringe 7 to alter the volume within an inner chamber 33 of the syringe 7. The plunger 23 of the syringe 7 is set such that 70 ml of air is present within the chamber 33 of the syringe 7.

FIG. 2B shows a representation of the cell sampling device once inserted into a rectal cavity. The plunger 23 of the syringe has been fully depressed which causes the flexible membrane 4 to inflate to a volume of 70 ml. The inflated flexible membrane 4 then makes contact with the colorectal mucosal surface of a human subject such that any exfoliated cells are transferred to the outer surface of the flexible membrane 4.

FIG. 2C shows a representation of the cell sampling device once exfoliated cells have been sampled and prior to removal from a rectal cavity. The plunger 23 of the syringe 7 is retracted such that 80 ml of air is present within the chamber of the syringe 7. This therefore creates a reduced pressure within the chamber which causes the flexible membrane 4 to be drawn back into the interior cavity of the insertion member 1 and adhere firmly to the side walls of the insertion member 1. The amount of reduced pressure may be pre-quantified by the presence of two snap fit retention features 24 (only one of which is shown in FIG. 2C). The snap fit features 24 are arms present on the plunger 23 of the syringe 7 which locate into holes on the barrel 32 of the syringe 7. The purpose of the snap fit features 24 is to prevent withdrawal of the plunger 23 from the syringe 7.

FIG. 2D shows a representation of the cell sampling device after removal from the rectal cavity and prior to cell analysis. The distal, insertion end of the insertion member 1 is provided with a thread which is adapted to receive a threaded screw cap 8. The cap 8 may have a blister packet containing a buffer such that upon screwing the cap 8 to the insertion member 1, the buffer is released into the receptacle formed by the deflated flexible membrane 4. After the cap 8 has been screwed to the insertion member 1, the syringe 7 may be detached from the insertion member 1 to allow the insertion member 1 to be converted to a compact assay vial which, along with a plurality of other vials, may be packaged and sent to a laboratory for cell analysis.

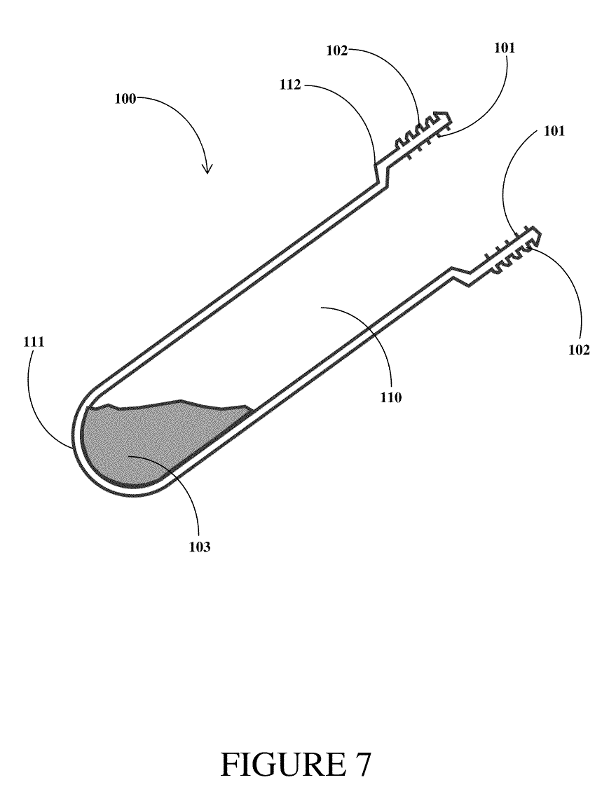

Alternatively the buffer can be placed in a double-threaded container shown in FIG. 7 that will be screwed on the insertion member. The syringe is then detached from the insertion member and the insertion member/container complex may be packaged and sent to a laboratory, where the insertion member is detached and discarded and the container is used as a storage or assay vial.

In one embodiment, the kit additionally comprises a closure device. In one embodiment, the closure device is a cap or lid having a threaded portion on an inner surface. In an alternative embodiment, the closure device is a snap-fit cap, lid, stopper or bung.

In one embodiment, the closure device is a snap-fit cap, lid, stopper or bung and the engagement means present on the container and/or cell sampling device comprise one or more protrusions configured to engage with the lid, stopper or bung in a snap-fit arrangement.

In another embodiment, a threaded portion may be present on the inner and outer surfaces of the container and the outer surfaces of the cell sampling device.

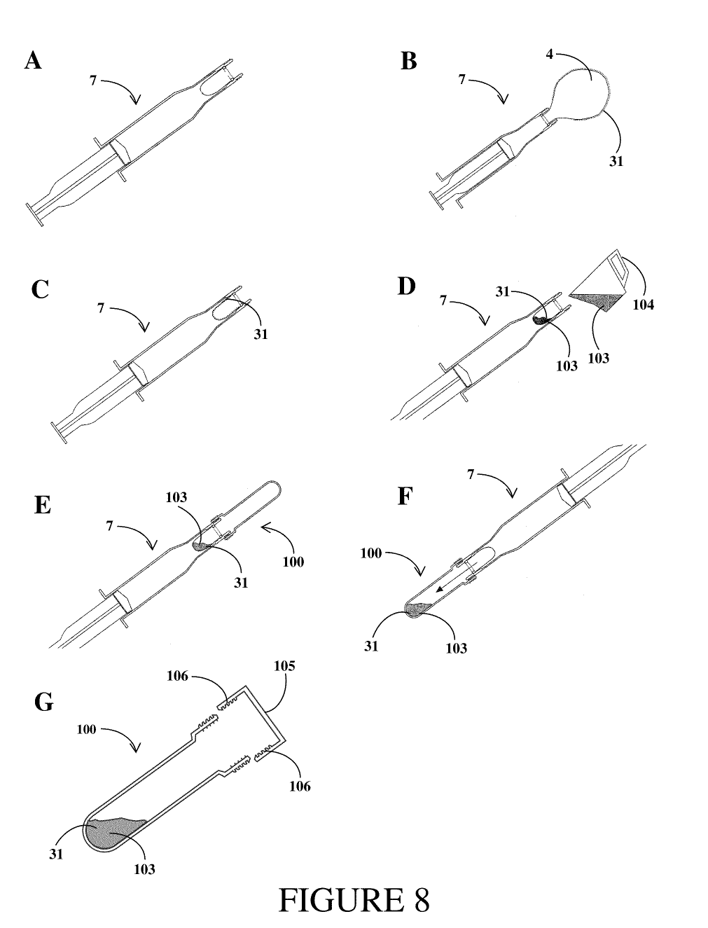

For example, a first threaded portion is present on the inner surface of the double-threaded container (e.g. an internal thread), a second threaded portion is present on its outer surface, and a third threaded portion is present on the outer surface of the cell sampling device (e.g. an external thread). This embodiment provides an arrangement wherein the container and cell sampling device may be attached to each other via the first and third threaded portions. In the first embodiment, a closure device for the container may be a stopper or bung or otherwise a cap or lid having a threaded portion on an inner surface (e.g. an internal thread) for sealing the container having the external thread. In the first embodiment, a closure device for the cell sampling device may be a cap or lid having a threaded portion on an inner surface (e.g. an internal thread) for sealing the cell sampling device having the external thread. This embodiment is shown in FIG. 7, and in operation in FIGS. 8e-8f.

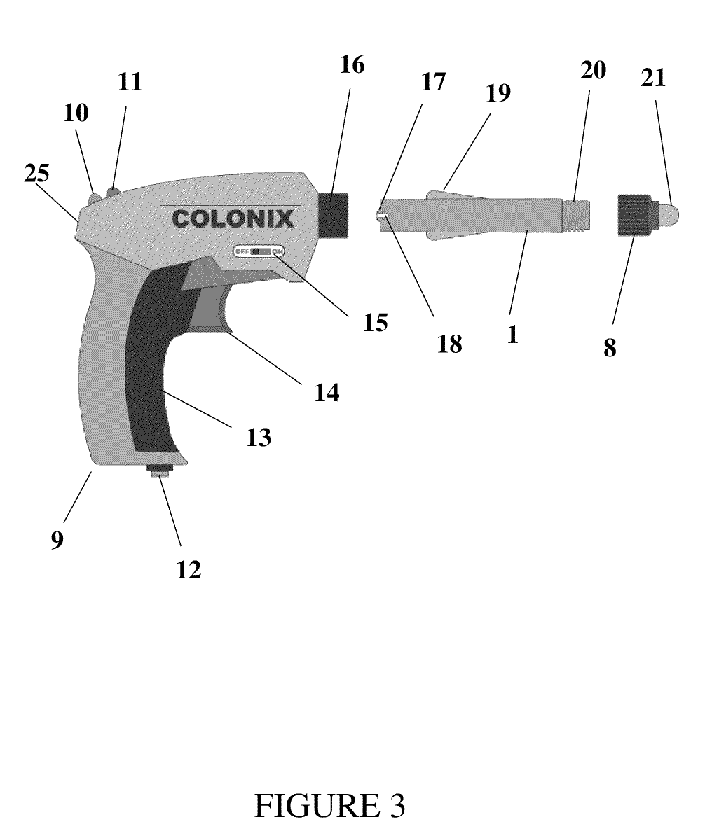

The embodiment of the invention wherein the means for pressurisation of the interior cavity 7 is a source of compressed air is shown in FIG. 3. This figure schematically shows a mechanical device 9 which is a pump operated by an electrical motor (not shown) capable of delivering repeated doses of a first elevated pressure followed by a second reduced pressure upon activation of the trigger 14. The mechanical device 9 is capable of attachment to an insertion member 1 substantially as described in FIGS. 1 and 2 by way of a click-fit locator 16 present on the mechanical device 9 which co-operates with a locating lug 17 on the insertion member 1. A self-sealing valve 18 is present on the insertion member 1 to ensure pressure is maintained within the insertion member 1 upon disconnection from the mechanical device 9. The insertion member 1 comprises vanes 19 which are designed to engage with a proctoscope and is threaded 20 at the distal insertion end in order to receive a threaded cap 8 which may contain a blister packet 21 containing the buffer. The buffer can alternatively be supplied in a double-threaded container with an inner thread and an outer thread. The mechanical device 9 is intended to be battery powered and may be re-charged by a power supply through a charging jack 12. The mechanical device 9 comprises an air intake filter 25, a rubberised handle 13 and also has an on-off switch 15 and light emitting diodes 10 and 11 which indicate when the device 9 is ready and when the cycle of first and second pressure applications are complete.

In use, a user holds the mechanical device 9 by the rubberised pistol type handle grip 13 and attaches the device 9 to an insertion member 1. The insertion member is then inserted into the rectal cavity where it engages with a proctoscope using the vanes 19 which enables an improved penetration consistency. A first elevated pressure is applied by the user by pressing the trigger 14 which causes air to be drawn into the mechanical device 9 through the air intake filter 25 which is then compressed and causes the flexible membrane to emit from the distal end of the insertion member 1 to make contact with the colorectal mucosal surface. A second reduced pressure is then applied by the user by pressing the trigger 14 a second time which causes the flexible membrane to return to the interior cavity of the insertion member 1. Once cell sampling has been completed, the insertion member 1 is disengaged from the proctoscope and the mechanical device 9 is detached from the insertion member 1 and the pressure within the insertion member 1 is maintained by way of the self-sealing valve 18. A threaded cap 8 having a buffer containing blister packet 21 may then be screwed to a thread 20 on the insertion member 1 causing buffer to be released into the receptacle formed by the deflated flexible membrane. The same effect can be achieved by a buffer containing double-threaded container. The mechanical device 9 can then be re-used by attachment to subsequent insertion members 1.

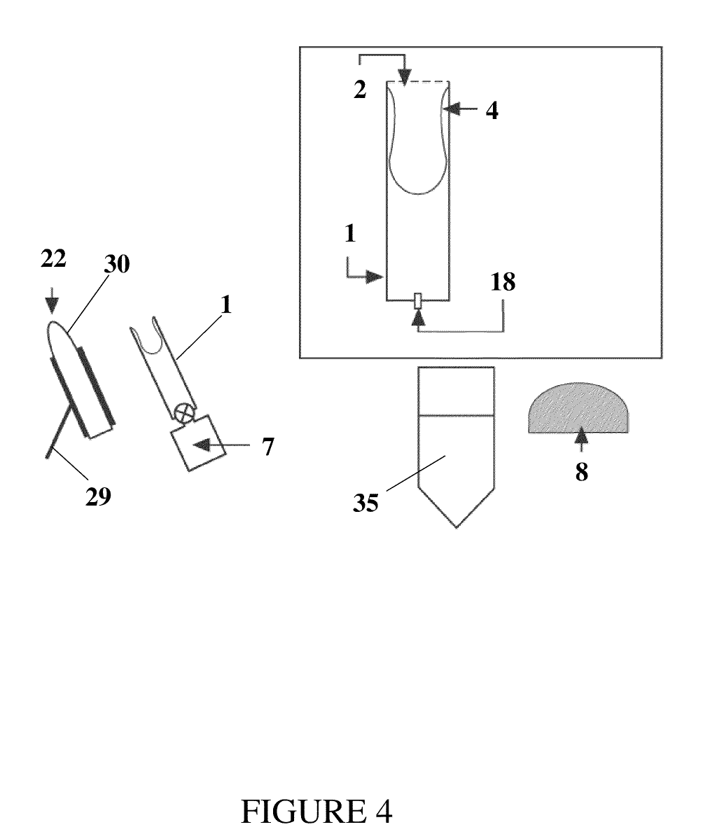

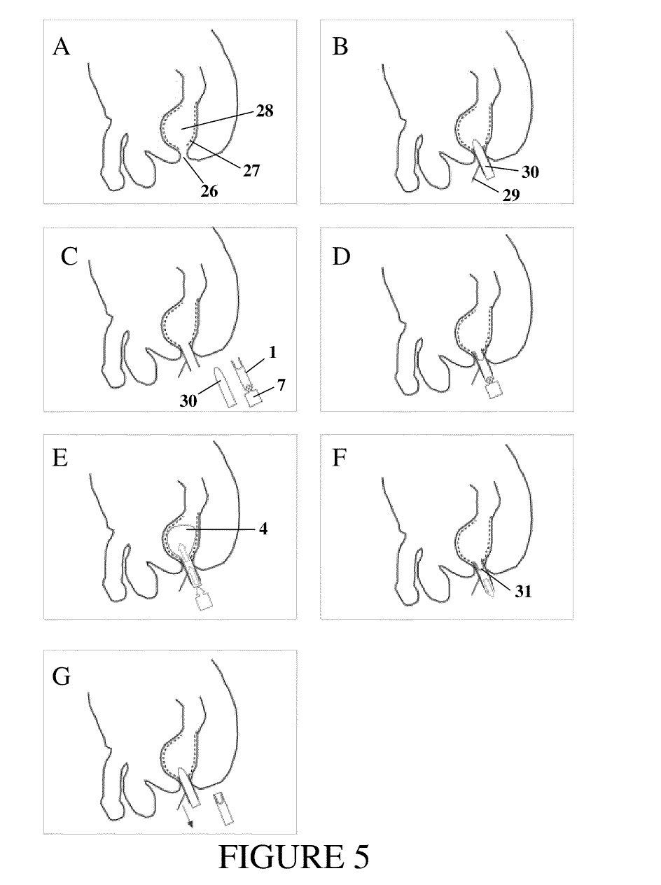

The components required for sampling exfoliated cells from a colorectal mucosal surface of a human subject are presented in FIG. 4.

Access to the rectal mucosa can be achieved by the use of a rectal access tube 29, which can be a modification of an existing instrument for rectal examination (e.g. rectoscope 22). The rectal access tube 29 consists of a rigid tube (with a handle) equipped with an obturator 30 providing an olive-shaped end and uninterrupted surface facilitating introduction of the rectal access tube 29 through the anal canal into the rectum.

The cell sampling device 1 shown in FIG. 4 is substantially as described in FIG. 1 and has an external diameter compatible with the internal diameter of the rectal access tube, i.e. in the range of 15-20 mm.

A source of compressed air 7 serves to provide a means for pressurisation of the interior cavity. The means for pressurisation 7 may comprise a syringe (as described in FIG. 2), an air pump (as described in FIG. 3) or a compressed air mini-container (mini-cylinder). Air pressure inside the cell-sampling device can be limited/controlled by either using a fixed air volume (simple syringe solution) or by reaching a fixed air pressure level (a precision valve would be needed for this purpose).

A bottle, tube or double-threaded container, with a specific buffer 35 (different buffers should be used for different purposes, such as DNA or RNA extraction or cell isolation/separation for further analysis).

A hermetic lid 8 for the cell-sampling device (needed for cell/protein lysis reactions if immediate DNA or RNA extraction is performed, for cell isolation procedures and, especially, for storage/transportation of the material if it is not immediately used, e.g. transportation from surgery/clinic to laboratory). In the embodiment shown in FIG. 8g, cap 105 is useful. after detaching the insertion member for long-term storage of the material in the laboratory

The components required for the procedure can be developed to be used as a disposable kit, which should include all the listed components except the compressed air source, which can be used repeatedly.

FIG. 5 shows an example of the cell sampling technique to sample exfoliated cells from a colorectal mucosal surface of a human subject using any of the devices shown in FIGS. 1-4. This procedure is simple and no special training in proctology or endoscopy is required for the operator to carry it out. It can be performed by any qualified medical professional (GP, nurse etc.) at a local surgery or patient's home or it may even be self-administered by the patient.

FIG. 5A schematically illustrates a cross-section of the anatomy of the human rectum 28, anal canal 26 and colorectal mucocellular layer 27. It should be noted that any contact of the cell-sampling device with squamous epithelium of anal canal can result in both material loss and contamination of the sample with squamous epithelium of the anal canal.

The procedure commences with introduction of a rectoscope-like rectal access tube 29 with the use of an obturator 30 in place in the rectum 28 (FIG. 5B). An appropriate lubricant can be used for the introduction procedure to facilitate it and to diminish patient's discomfort, which can be caused by this initial stage of the procedure.

Once the rectal access tube 29 is introduced (FIG. 5C) and the obturator 30 has been removed, direct access to rectal mucosa is achieved and the mucocellular layer 27 opens.

The insertion member 1 is introduced to the rectal access tube 29 so that the upper edge of the insertion member is located just above the edge of the rectal access tube (FIG. 5D).

A first elevated pressure is applied which inflates the collecting flexible membrane in order to contact the membrane with the rectal mucocellular layer 27 to provide exfoliated cell sampling (FIG. 5E). The device is left in this position for approximately 10-15 seconds to achieve better adhesion of exfoliated cells and cell-derived materials of the mucocellular layer to the collecting membrane.

FIG. 5F shows the application of a second reduced pressure which deflates the flexible membrane and causes it to return to its initial position with collected material 31 on the outer, cell sampling surface.

The insertion member 1 is removed from the rectal access tube 29 and taken for further manipulations and analyses. The obturator 30 (a new re-lubricated one can be used) is optionally reinstalled into the rectal access tube 29, and the tube 29 is removed from the rectum 28 (see FIG. 5G). The complete procedure (rectal manipulations) should take no more than a couple of minutes.

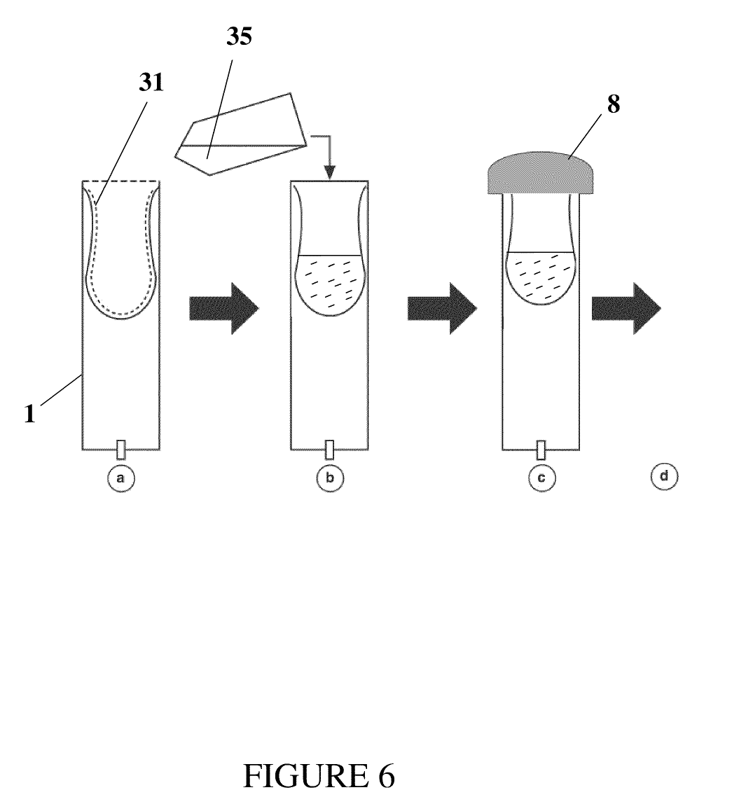

FIG. 6 shows an example of the steps which may follow the method depicted above (in FIGS. 5A-5G) which should be completed immediately after cell collection to avoid drying of the cell collection membrane. Step (a) shows cell-sampling device 1 with exfoliated cells 31 on the cell-collecting flexible membrane after cell collection. The top compartment of the cell-sampling device is filled with a fixed volume of a specific buffer 35 which either lyses or suspends and preserves the exfoliated cells (Step (b)). The lysate or cell suspension can be left in the complex of the insertion member and the double-threaded container 100, shown in FIGS. 7 and 8e-8f for transport. Different cell lysis buffers or cell preserving mediums can be used for DNA or RNA extraction procedures, special buffers/mediums should be used for applications requiring protein analysis or cell isolation.

The cell-sampling device with collected material is prepared for sample transport or storage by being hermetically closed with a secure threaded cap 8 (step (c)) but it will be appreciated that when the threaded cap has a buffer containing blister packet then step (b) can be omitted. Alternatively the collected material can be transported or stored in a complex of the insertion member and the double-threaded container. The device can then be stored or transported for further downstream procedures for screening/diagnostic and/or research purposes (step (d)).

To perform a direct quantification of the amount of DNA extracted from the cells, the initial buffer used just after cell sampling should be a cell lysis buffer used for the selected DNA extraction procedure. The addition of the buffer should provide efficient cell lysis and preservation of the DNA-containing material during transportation to a dedicated laboratory and for some period of storage

Optionally, prior to the introduction of the lysis buffer, a small fraction of the collected material may be used to prepare smears on microscope slides for cytological examination which can provide useful diagnostic information.

The DNA extraction method should be selected on the basis of its applicability for high throughput analysis, i.e. it should be compatible with multichannel liquid handling robotic systems.

Similar initial steps of DNA extraction can be applied for the analysis of molecular markers of colorectal cancer. Cells sampled by this method should provide a much better quality DNA compared to currently employed techniques of DNA extraction from stool samples. PCR amplification of this DNA can be done without precise quantification of its amount. Multi-target molecular analysis is considered as an option in colorectal cancer screening, however it may be more time-consuming and expensive compared to direct quantitative analysis. At the same time DNA extracted for direct quantification can certainly be used for PCR amplification in further diagnostic analysis of quantitatively “positive” or “doubtful” cases.

In case of a need for specific analysis of colonocytes or cells of other types (e.g. free cells such as neutrophils, lymphocytes, macrophages etc), separation methods (e.g. immunomagnetic or density gradient separation) can be applied to achieve a higher purity of cell populations for the analysis. For this purpose some cell-preserving media containing antibiotics (some bacterial presence in the collected material is impossible to avoid), protease inhibitors protecting from protein degradation and mucolytic agents can be applied. Isolated colonocytes can then be used for different types of analysis such as DNA extraction and quantification, DNA extraction followed by PCR amplification, RNA isolation, protein assays, cancer molecular and biochemical marker analysis, cytological/cytochemical assessment, and direct cell counting (doubtful in terms of screening due to low speed and high cost).

Colorectal Cancer Detection Method

Once the sample containing the lysate has reached the laboratory, it can be removed from the sampling device for analysis.

Prior to performing the DNA assay, it may be desirable to determine the level of faecal contamination of the sample to determine if the sample is viable for purposes of performing the DNA assay. Faecal contamination can be assessed by measuring the optical absorbance of approximately 100 μl of the lysate at a wavelength of 340 nm using a fluorescent microplate reader or other similar equipment. In one embodiment of the invention, contamination degrees can be defined as follows: less than 1.5, low or acceptable; 1.5 or higher, high or unacceptable (values in optical absorbance units). As will be realized by one of skill in the art, any other well-known method of determining faecal contamination may be used.

Two methods of performing the DNA assay are provided, the first will be referred to herein as the “PicoGreen method” and the second as the “QRTPCR (Quantitative Real Time Polymerase Chain Reaction) method”. However, other methods well known in the art may be substituted. In the preferred embodiment, the PicoGreen method seems to provide better results, providing higher DNA scores and better detection rate for proximally located tumour than the QRTPCR method.

The PicoGreen Method

For quantification using the PicoGreen method, DNA can be isolated from as little as 120 μl of lysate using a commercially available QIAamp DNA mini kit. DNA concentrations can be measured using an Invitrogen-iT™ PicoGreen® dsDNA assay kit and a fluorescent microplate reader at wavelengths of 485 nm (excitation) and 535 nm (emission) according to the instructions provided with the kit. The final value or DNA score reflects DNA concentration in units of μg/ml recalculated for the original lysate. Alternative fluorescent dyes such as Hoechst 33258 can be used for this purpose. Direct DNA concentration determination by measuring UV absorbance at 260 and 280 nm can also be applied, however reliable results can only be obtained if larger initial lysate volumes are used in order to achieve higher DNA concentrations after purification (UV260/280 assay has much lower sensitivity).

The QRTPCR Method

Using the RT-PCR method, amplifiable human DNA in the samples can be quantified by targeting a human-specific 71-bp fragment of the β-globin gene (exon 3) with a known set of PCR primers and a TaqMan probe:

forward primer,
5′-GGGCAACGTGCTGGTCTG-3′;
reverse primer,
5′-AGGCAGCCTGCACTGGT-3′;
TaqMan probe,
5′-FAM-CTGGCCCATCACTTTGGCAAAGAA-BHQ-3′
(FAM - 6-carboxyfluorescein; BHQ, Blackhole
Quencher 1).

The 15 μl reactions may include 1× QuantiFast Probe PCR master mix, both primers at 400 nm, the TaqMan probe at 200 nm and 6 μl of tested sample. Amplifications can be performed using a system suitable for quantitative real time PCR with pre-incubation at 95° C. for 3 min followed by 40 cycles of denaturation at 95° C. for 3 sec and annealing/extension at 60° C. for 30 sec. Amplifiable DNA concentrations can be calculated by extrapolation from calibration curves generated by QRTPCR of fixed amounts of human genomic DNA. The DNA concentrations are automatically transformed into DNA scores.

DNA extraction followed by PCR amplification and molecular analysis can be useful both for confirmation of the initial diagnosis and for advanced diagnostic procedures (assessment of cancer aggressiveness, sensitivity to chemotherapy for metastatic tumours, prognosis etc.).

Cell isolation can be used for both further molecular or biochemical analysis and cytological investigation (tumour cells with specific morphological features) can be easily found among exfoliated colonocytes in CRC patients.

Statistical Analysis of Results.

To determine the proper cut off points for determining the demarcation between positive and negative results, a statistical baseline model has been established based on empirical data.

A study was conducted to establish the statistical baseline. Two groups of individuals were recruited, the first group consisting of 110 clinically healthy volunteers. Any prospective volunteers reporting colorectal symptoms, colorectal disease history or family history of CRC were not included in the study.

The second group consisted of 66 consecutive patients with a clinical diagnosis of CRC. Cases of low rectal tumours detectable by digital rectal examination were not included in the study. All samples were collected at least a few days before planned operations. No bowel preparation was used in either group.

TABLE 1
Characteristics of study subjects and collected Samples.

| | Number of subjects | | | |
|---|---|---|---|---|
| | Healthy volunteers | | CRC patients | |
| | Total | Eligible | Total | Eligible |
| Variable | 110 | 101 | 66 | 56 |
| Sample contamination degree: | | | | |
| Low | 102 (92.7%) | 96 (95.0%) | 62 (93.9%) | 53 (94.6%) |
| Heavy | 8 (7.3%) | 5 (5.0%) | 4 (6.1%) | 3 (5.4%) |
| Samples excluded: Total | 9 | | 10 | |
| For technical reasons | 4 | | 3 | |
| Because of non-compliance | 5 | | 7 | |
| Diagnoses: | | | | |
| Large polyps (≧10 mm) | | | 2 | 1 |
| CRC (total) | | | 58 | 55 |
| CRC site - Caecum | | | 7 | 7 |
| CRC site - Ascending Colon | | | 8 (10) | 7 (9) |
| CRC site - Hepatic Flexure | | | 2 | 2 |
| CRC site - Transverse Colon | | | 2 | 2 |
| CRC site - Descending Colon | | | 1 | 1 |
| CRC site - Sigmoid colon | | | 13 (14) | 12 (13) |
| CRC site - Rectum | | | 26 | 25 |
| Other conditions (non-CRC) | | | 6 | — |

There was no opportunity to apply endoscopic investigation to the volunteers of the control group; therefore these asymptomatic subjects were regarded as clinically healthy. Most of the CRC patients eventually had operations, and post-operative pathology reports were used as the final diagnosis. All tumours distal to the splenic flexure (the descending colon, the sigmoid colon and the rectum) were classified as distal and all remaining tumours (the caecum, the ascending colon and the transverse colon) as proximal. A summary of the subjects participating in the study and their conditions is shown in Table 1.

Initial analysis of DNA scores included descriptive statistics (means, 95% confidence intervals, t-tests) for the control and case groups as well as for subgroups of proximal and distal tumour cases taken separately.

To approach the selection of DNA score cut-off point separating negative (likely disease-free) and positive (likely disease-affected) results, test receiver operating characteristic (ROC) curves were analyzed for the following situations:
controls vs. all CRC cases (proximal+distal tumours);
controls vs. proximal colon cancer cases only;
controls vs. distal CRC cases only.

Sensitivity and specificity for detecting CRC at different cut-off points were expressed as percentages, with sensitivity defined as the proportion of subjects with the disease who tested positive and specificity defined as the proportion of disease-free subjects who tested negative. Ninety-five percent confidence intervals (CIs) were calculated for the proportions using the exact probabilities of the Binomial distribution.

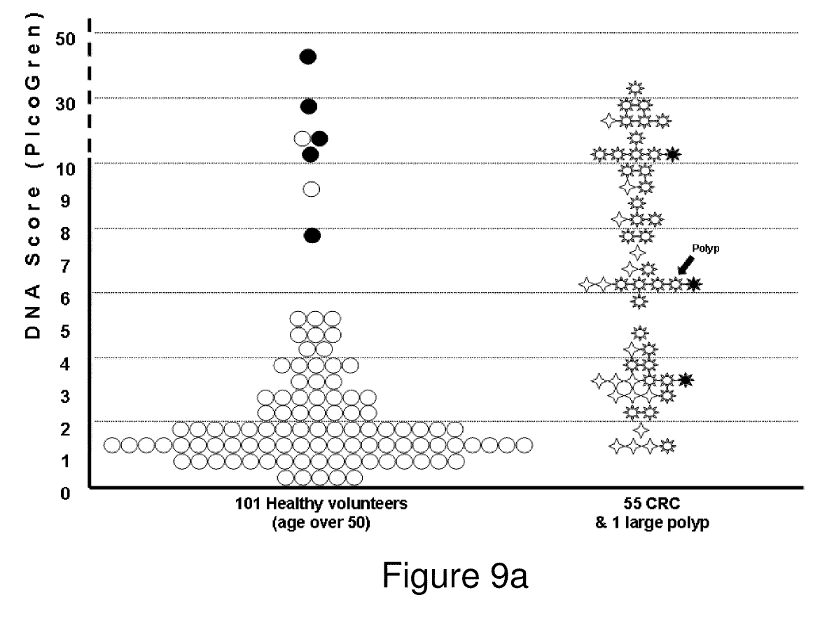

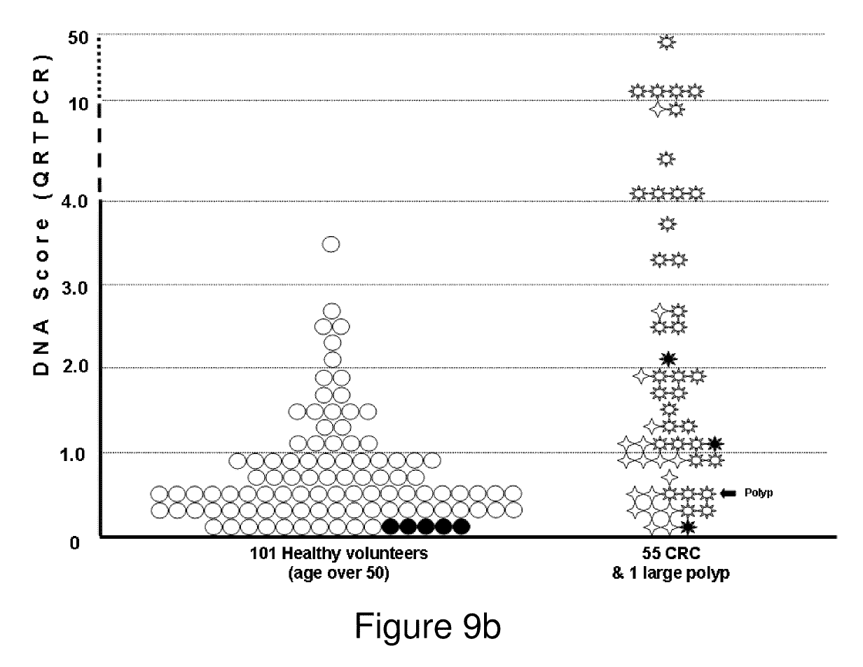

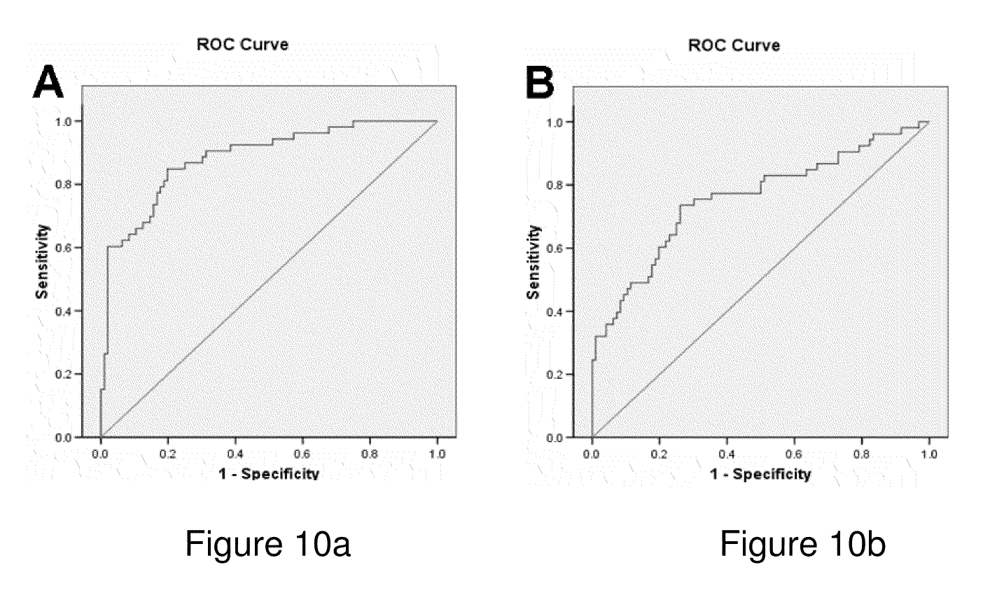

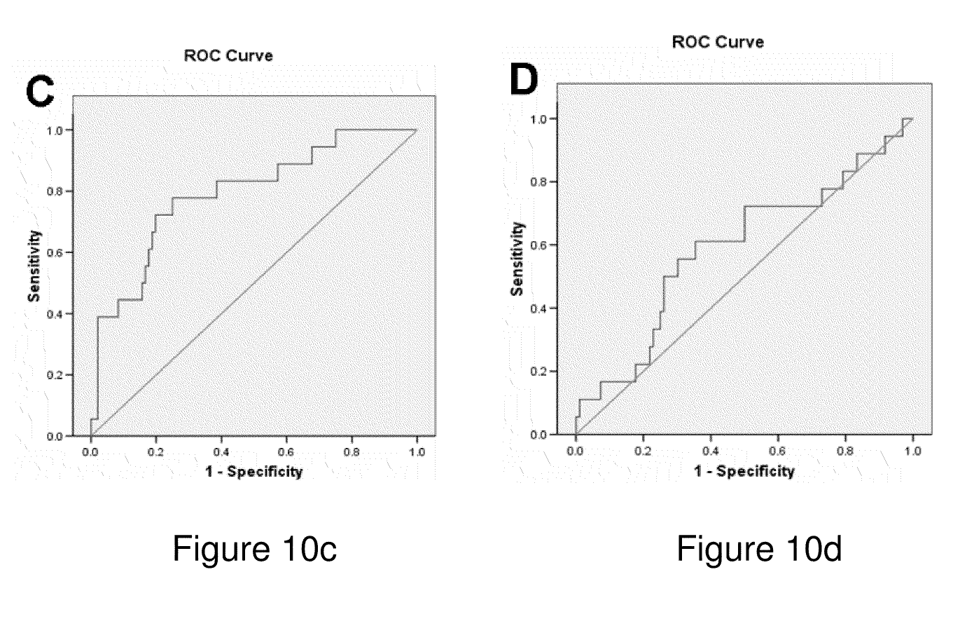

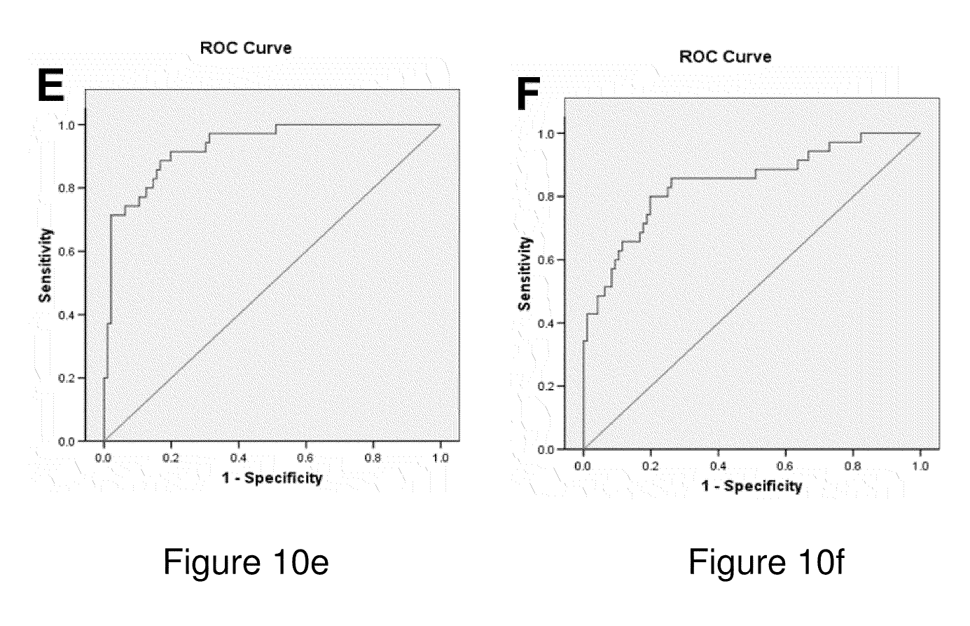

Although DNA scores obtained by the PicoGreen assay were higher than those generated by QRTPCR, result distribution patterns looked similar for the two assays (See FIGS. 9a & 9b). In these figures, circles indicate individual results in healthy volunteers; four-pointed stars—proximal cancer cases; eight-pointed stars—distal cancer cases; White symbols indicate low faecal contamination of samples; black—heavy faecal contamination of samples.

The difference between the methods was evident for heavily contaminated samples. High contamination was associated with higher PicoGreen DNA scores and much lower QRTPCR scores. This discrepancy was an additional reason for excluding heavily contaminated samples from further analysis.

As shown in Table 2, in the control group DNA scores were mostly low and did not depend on either sex or age of volunteers (comparison results are not shown). Mean DNA scores obtained by the both methods in the whole CRC group were significantly higher.

TABLE 2
DNA scores in clinically healthy volunteers and colorectal
cancer patients determined by PicoGreen (PG) assay
and quantitative real-time PCR (QRTPCR).

| | | Mean | | | |
|---|---|---|---|---|---|
| | | DNA | | P-value | P-value |
| Study groups | Assay | (μg/ml) | 95% CI | (vs control) | (proximal vs |
| Control | PG Assay | 2.1 | 1.7-2.5 | — | — |
| (96 healthy | QRTPCR | 0.8 | 0.6-0.9 | — | — |
| volunteers) | | | | | |
| All CRC | PG Assay | 9.0 | 6.7-11.2 | <0.001 | — |
| (53 cases, | QRTPCR | 3.8 | 1.9-5.7 | 0.003 | — |
| proximal + | | | | | |
| distal) | | | | | |
| Proximal | PG Assay | 5.2 | 3.0-7.4 | 0.014 | 0.005 |
| colon | QRTPCR | 1.4 | 0.3-2.4 | 0.275 | 0.018 |
| cancer | | | | | |
| (18 cases) | | | | | |
| Distal colon | PG Assay | 10.9 | 7.8-14.0 | <0.001 | 0.005 |
| cancer | QRTPCR | 5.1 | 2.3-7.8 | 0.004 | 0.018 |
| (35 cases) | | | | | |

Comparison of proximal and distal CRC showed that mean DNA scores for distal tumours were significantly higher than for proximal ones (Table 2). Nevertheless, the mean PicoGreen DNA score for proximal cancers was still significantly higher than in the control group. There was no statistically significant difference between proximal cancer patients and controls if QRTPCR was applied. QRTPCR DNA scores were below 1.0 μg/ml for most cases of proximal tumours (See FIG. 9b). Mean DNA scores for distal CRC obtained by the both assays were significantly higher than in the control group.

DNA scores in the CRC group were not affected by the Dukes' stage, tumour size or tumour histopathology. Blood presence in some samples did not seem to influence DNA score results either.

ROC curves were examined to assess the efficiency of CRC detection by the test. This analysis was performed for all CRC cases pooled together and for proximal and distal tumours separately (FIG. 10). Sensitivity and specificity as well as positive and negative predictive values and likelihood ratios for the PicoGreen and QRTPCR assays at several tentative DNA score cut-off points are presented in Tables 3 and 4 respectively.

Comparison of panels A and B of FIG. 10 shows that the PicoGreen assay identified CRC cases more efficiently, the main reason for the difference being poorer QRTPCR performance for proximal tumours (FIG. 10D). Both distal and proximal cancers were reliably detected by the PicoGreen assay (areas under the ROC curve 0.93 and 0.79 respectively), but test sensitivity and specificity values depended on the selected cut-off point (Table 3). While the sensitivity for detecting distal CRC was still 71.4% at the cut-off point of 5.0, providing the specificity of 94.8%, the majority of proximal cancers became undetectable at this high cut-off point. At the same time Pico Green cut-off point of 2.5 provided high sensitivity for both proximal and distal tumours (77.8% and 91.4% respectively), whereas the specificity decreased to 74.0%.

TABLE 3
Test sensitivity, specificity, positive and negative predictive
values, and likelihood ratios for the PicoGreen-based assay
at different cut-off points (DNA scores demarcating positive
and negative results).

| | Sensitivity | | | |
|---|---|---|---|---|
| | | For proximal | For distal | |
| | For all CRC | tumour | tumour | |
| | cases, % | cases, % | cases, % | Specificity % |
| Cut-off point | (95% CI) | (95% CI) | (95% CI) | (95% CI) |
| 2.0 | 90.6 | 77.8 | 97.1 | 66.7 |
| | (79.3-96.7) | (52.4-93.6) | (85.1-99.9) | (56.3-76.0) |
| 2.5 | 86.8 | 77.8 | 91.4 | 74.0 |
| | (74.7-94.5) | (52.4-93.6) | (76.9-98.2) | (64.0-82.4) |
| 3.0 | 79.2 | 61.1 | 88.6 | 81.2 |
| | (65.9-89.2) | (35.7-82.7) | (73.3-96.8) | (72.0-88.5) |
| 3.5 | 69.8 | 44.4 | 82.9 | 84.4 |
| | (55.7-81.7) | (21.5-69.2) | (66.3-93.4) | (75.5-91.0) |
| 4.0 | 66.0 | 44.4 | 77.1 | 89.6 |
| | (46.0-73.5) | (21.5-69.2) | (59.9-89.6) | (81.7-94.9) |
| 4.5 | 64.2 | 44.4 | 74.3 | 91.7 |
| | (49.8-76.9) | (21.5-69.2) | (56.7-87.5) | (84.2-96.3) |
| 5.0 | 60.4 | 38.9 | 71.4 | 94.8 |
| | (46.0-73.5) | (17.3-64.3) | (53.7-85.4) | (88.3-98.3) |
| Area under | 0.88 | 0.79 | 0.93 | |
| the ROC | (0.83-0.94) | (0.68-0.91) | (0.89-0.98) | |
| curve | | | | |

TABLE 4
Test sensitivity, specificity, positive and negative predictive
values, and likelihood ratios for the quantitative real-time
PCR-based assay at different cut-off points (DNA scores
demarcating positive and negative results).

| | Sensitivity | | | |
|---|---|---|---|---|
| | | For proximal | For distal | |
| | For all CRC | tumour | tumour | |
| | cases, % | cases, % | cases, % | Specificity % |
| Cut-off point | (95% CI) | (95% CI) | (95% CI) | (95% CI) |
| 0.50 | 83.0 | 72.2 | 88.6 | 40.6 |
| | (70.2-91.9) | (46.5-90.3) | (73.3-96.8) | (30.7-51.1) |
| 0.75 | 77.4 | 61.1 | 85.7 | 60.4 |
| | (63.8-87.7) | (35.7-82.7) | (69.7-95.2) | (49.9-70.3) |
| 1.00 | 64.2 | 33.3 | 80.0 | 76.0 |
| | (49.8-76.9) | (13.3-59.0) | (63.1-91.6) | (66.2-84.2) |
| 1.25 | 54.7 | 22.2 | 71.4 | 82.3 |
| | (40.4-68.4) | (6.4-47.6) | (53.7-85.4) | (73.2-89.3) |
| 1.50 | 49.1 | 16.7 | 65.7 | 87.5 |
| | (35.1-63.2) | (3.6-41.4) | (47.8-80.9) | (79.2-93.4) |
| 1.75 | 43.4 | 16.7 | 57.1 | 90.6 |
| | (29.8-57.7) | (3.6-41.4) | (39.4-73.7) | (82.9-95.6) |
| 2.00 | 39.6 | 11.1 | 51.4 | 92.7 |
| | (26.5-54.0) | (1.4-34.7) | (34.0-68.6) | (85.5-97.0) |
| Area under | 0.76 | 0.59 | 0.84 | |
| the ROC | (0.67-0.84) | (0.44-0.74) | (0.76-0.93) | |
| curve | | | | |

As there was no chance of examining the control group endoscopically, the presence of asymptomatic colorectal conditions in some of its members could not be completely excluded. Moreover, no dietary or lifestyle advice was given to the participants of the study; hence diet or alcohol ingestion could potentially influence the results. Although volunteers did not provide complete information on their diet and drinking habits, nine individuals reported alcohol consumption within 24 hours before material collection. Mean PicoGreen and QRTPCR DNA scores in these nine people (2.7 μg/ml and 1.0 μg/ml respectively) were slightly higher than control group means (see Table 2 for comparison).

The results demonstrate that the test is highly sensitive for detecting distal CRC. The sensitivity for proximal cancers was lower, but it should be stressed that these tumours are notoriously difficult to diagnose even by colonoscopy. Hence the sensitivity of 77.8% obtained at the DNA score cut-off point of 2.5 μg/ml (PicoGreen assay) could improve proximal tumour detection. It is, however, obvious that lowering the cut-off point in order to achieve better detection of the proximal cancers decreases test specificity. The latter consideration is important for a potential use of the test for CRC screening since high specificity is one of the obligatory requirements for a screening test.

Application of a much higher cut-off point set at 5.0 μg/ml (PicoGreen assay) results in almost 95% test specificity, but sensitivity values (see Table 3) become close to those achieved by recent versions of immunochemical FOBT. Additional standardization measures discussed below can improve test specificity at lower cut-off points.

The results of the DNA assay by the two methods showed that differences between the very low DNA scores observed in the tumour-free subjects and relatively higher scores detected in subjects having CRC are highly statistically significant.

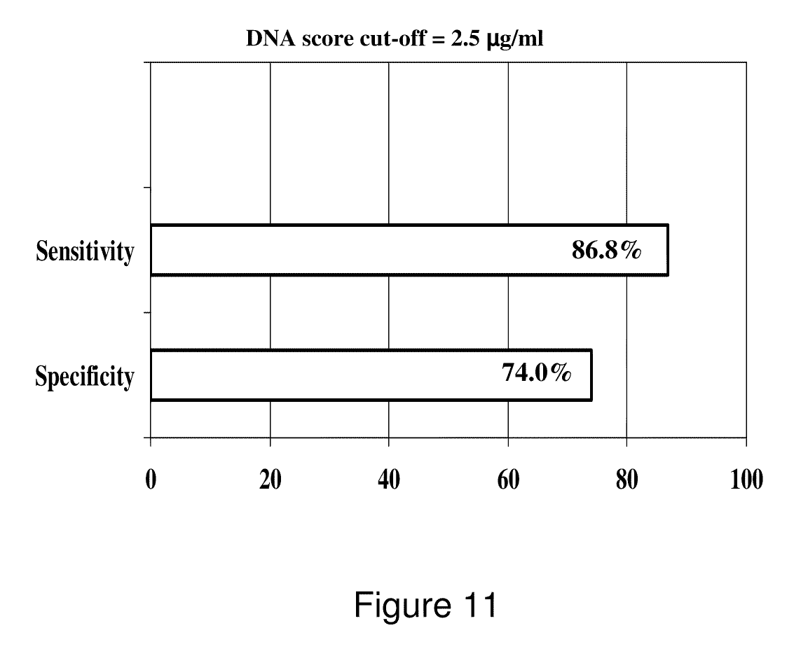

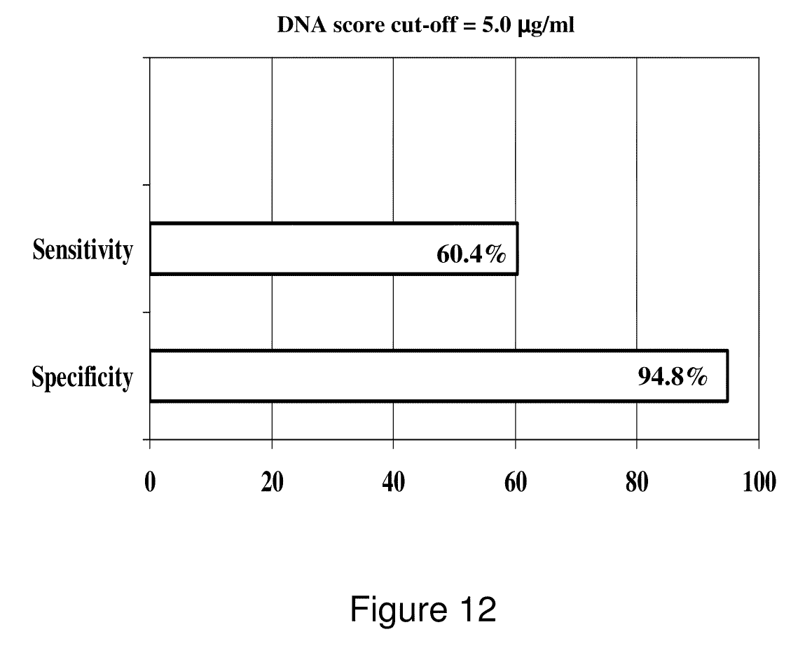

FIGS. 11 and 12 show specificity and sensitivity percentages for cut off points of 2.5 μg/ml and 5.0 μg/ml respectively. For screening purposes high test specificity is desirable, therefore, in one embodiment of the invention, a cut off point of between 3.0 μg/ml and 5.0 μg/ml will likely be selected as the demarcation between a positive and a negative result. In the preferred embodiment, the cut off point will be around 4.0 μg/ml

It is expected that advising the patient to avoid food within 6 hours of the test will result in a higher specificity by avoiding faecal contamination and in a higher sensitivity as a result of the no bowel movements being made prior to the test. Additionally, advising the patient that alcohol should be avoided 24 hours prior to the test should also help to increase the specificity by avoiding stimulated cell exfoliation. It has also been discovered that collecting material within two hours following bowel opening is not advisable since: a) faecal contamination is high in about 40% of samples collected within this period; b) test result is likely to be unreliable since abundant material usually present in the rectal cavity of a patient with CRC is likely to be evacuated during defecation, and time is needed for its accumulation after that. Therefore, no samples should be collected within two hours following a bowel movement.

The test may be used for two distinct purposes within colorectal cancer detection, the first being for screening of asymptomatic groups of individuals within general population known to have an elevated CRC risk (e.g. people over 50), and the second being for optimising diagnostic routines (e.g. avoiding unnecessary colonoscopies).

For CRC screening purposes a high test specificity (at least about 95%) is required. Therefore, a relatively high cut-off point should be selected, one that correlates with a 95% specificity. Current results show that a cut-off of 4.0 μg/ml yields a specificity of 89.6%. Additional standardization measures are likely to bring it up to the required standard, therefore 4.0 μg/ml can be recommended as the cut off point for CRC screening.

For purposes of optimising diagnostic routines, the test can also be used as a “pre-colonoscopy test” to assist clinicians in assessing the necessity of urgent further investigation (in particular conventional or virtual colonoscopy) in symptomatic patients with relatively mild or unclear complaints, which may or may not be related to serious colorectal conditions, especially CRC (such as blood in stool, bowel habit changes, anemia of unknown origin etc). These patients are usually sent to colonoscopy, but the vast majority of them are then found to be tumour-free, so colonoscopies done because of such symptoms are often unnecessary. The test, when used in this manner, can be used to rank patients according to their relative risk of having cancer (or other serious condition such as severe inflammation). I the case case-control study performed to date, the pattern shown in Table 5 has emerged. Although the observed pattern graphically illustrates the principle of the approach in terms of the incidence trend, large number of cancer cases in the study gives greatly exaggerated estimates of CRC incidence.

TABLE 5
CRC incidence within DNA score ranges in a case-control study
including healthy volunteers and CRC patients.

| | | Number of observations |
|---|---|---|
| DNA Score | CRC Incidence | (Number of CRC cases) |
| <1.0 | 0.00% | 22 (0) |
| 1.0-2.0 | 8.70% | 46 (4) |
| 2.0-4.0 | 34.30% | 35 (12) |
| 4.0-7.0 | 57.90% | 19 (11) |
| Over 7.0 | 91.70% | 24 (22) |

The following reasoning can be applied, based on these scores:
DNA score is over 7.0: urgent actions required;
DNA score between 4.0 and 7.0: the patient needs to be sent for further detailed investigation;
DNA score between 2.0 and 4.0: some period of monitoring (probably including a repeated test) can be allowed, thus the decision on the necessity of further investigation can be postponed
DNA score between 1.0 and 2.0: a prolonged period of observation is acceptable with a possibility of releasing the patient if symptoms disappear (a common situation); and
DNA score below 1.0: the patient can be released, but will be advised to come back if symptoms persist.

The main distinctive feature of the “pre-colonoscopy” version of the test is that here there is no single cut-off point, but consecutive DNA score ranges form a scale of probabilities for a patient having CRC within each range.

In yet another embodiment of the invention, certain ranges of DNA scores within the statistical baseline for various types of patients have been discovered, with a major factor being tumour location, either distal or proximal. The statistical baseline appears to have well-defined ranges therein to discriminate between these conditions, and to provide cut off points to demark between healthy subjects, subjects likely having proximal CRC and subjects likely having distal CRC. Therefore, the test may also be useful in diagnosing proximal or distal tumours.

One skilled in the art will realize that different measuring methods and scales may be applied that will yield different actual numbers for the DNA scores, and that the scale and cut-off points to demark between positive and negative results and to define the location ranges would need to be adjusted accordingly.

We claim:

1. A method of screening for colorectal cancer, comprising the steps of:
a. performing DNA quantification on a sample consisting of a lysate containing colonocytes collected from the rectal mucosa of an individual;
b. calculating a DNA score based on said DNA quantification;
c. comparing said DNA score to a statistical baseline; and
d. returning a positive result when said DNA score exceeds a pre-determined cut off point established in said statistical baseline.

2. The method of claim 1 wherein said colonocytes have been collected by an inflatable collection membrane in direct contact with the rectal mucosa of said individual.

3. The method of claim 2 wherein said inflatable membrane has been deflated and stored inside a rigid body, and further wherein a buffer has been introduced into said deflated membrane to form said lysate.

4. The method of claim 1 further comprising the step, prior to said quantification step, of assessing said lysate for faecal contamination, and rejecting said sample if the degree of faecal contamination exceeds a predetermined level.

5. The method of step 4 wherein said degree of faecal contamination is measured by measuring optical absorbance.

6. The method of claim 5 wherein said lysate is exposed to a light of wavelength 340 nm and rejecting said sample when the absorbed light equals or exceeds 1.5 optical absorbance units.

7. The method of claim 1 wherein said step of DNA quantification further comprises the steps of:
a. isolating DNA from said lysate; and
b. measuring the concentration of DNA utilizing a PicoGreen assay method.

8. The method of claim 7 wherein said pre-determined cut off point is selected to correlate with test specificity of at least 95% for CRC screening purposes.

9. The method of claim 1 wherein said step of DNA quantification further comprises the steps of:
a. identifying a human-specific fragment of a gene;
b. amplifying said gene fragment utilizing a complimentary primer fragment; and
c. calculating the concentration of DNA by extrapolating said amplified gene fragment from known calibration curves.

10. The method of claim 8 wherein said human-specific gene fragment is a 71-bp fragment of the β-globin gene.

11. The method of claim 1 further comprising the step of determining the location of said colorectal cancer based on the DNA quantification.

12. The method of claim 11 wherein said location of said colorectal cancer is classifying as being located either in the proximal or distal colon.

13. The method of claim 2 wherein said sample is collected utilizing a sampling device consisting of:
a. a colorectal insertion member having a distal, insertion end, a proximal end and a closable interior cavity;
b. a flexible membrane having an outer, cell sampling surface and an inner surface, wherein said membrane is sealingly attached to the distal, insertion end of said insertion member and held within the interior cavity;
c. such that, in use, pressurisation of the interior cavity to at least a first elevated pressure causes the membrane to emit from the distal end of said insertion member to make contact with the colorectal mucosal surface and pressurisation of the interior cavity to a second reduced pressure causes the membrane to invert and return to the interior cavity of said insertion member.

14. A method of screening for colorectal cancer, comprising the steps of:
a. receiving a flexible membrane having an cell sampling surface forming a cavity, said cavity filled with a buffer forming a lysate containing exfoliated cells from the rectal mucosa of a subject;
b. performing DNA quantification on said lysate to determine the concentration of said DNA;
c. returning a positive or negative result based on the level of said concentration of DNA in said lysate.

15. The method of claim 16 wherein step (c) further comprises the steps of:
a. calculating a DNA score based on said DNA quantification;
b. comparing said DNA score to a statistical baseline; and
c. returning said result based on said comparison.

16. The method of claim 17 wherein step (c) further comprises the step of returning a positive result when said DNA score exceeds a pre-determined cut off point established in said statistical baseline.

17. The method of claim 16 further comprising the step of predicting the location of said colorectal cancer based on said DNA score.

18. The method of claim 17 wherein said statistical baseline contains a plurality of ranges for said DNA score, said ranges consisting of
a. a first range indicating no colorectal cancer;
b. a second range indicating the presence of colorectal cancer either proximal or distal colon; and
c. a third range indicating the presence of colorectal cancer most probably in the distal colon.

19. A method of detecting colorectal cancer, comprising the steps of:
a. performing DNA quantification on a sample consisting of a lysate containing colonocytes collected from the rectal mucosa of an individual;
b. calculating a DNA score based on said DNA quantification;
c. comparing said DNA score to a statistical baseline, wherein said statistical baseline defines ranges of DNA scores indicating differing results; and
d. advising a course of an action for applying further diagnostic procedures based on the range into which said DNA score falls.

20. The method of claim 19 wherein said ranges of DNA scores correlate to the likelihood that colorectal cancer is present.
